(19) United States
(12) Patent Application Publication
(10) Pub. No.: US 2006/0293258 A1
(43) Pub. Date: Dec. 28, 2006

(54) Method and composition to treat skin ulcers

(76) Inventor: Peter Rohdewald, Munster (DE)

Correspondence Address: Mintz, Levin, Cohn, Ferris, Glovsky & Popeo, P.C.;The Chrysler Center, 666 Third Avenue, 24th Floor, New York, NY, 10017, US

(21) Appl. No.: 11/166,510

(22) Filed: Jun. 23, 2005

(51) Int. Cl. A61K 31/7048; A61K 31/353; A61K 9/48

(52) U.S. Cl. 514/27; 514/456; 424/451

(57) ABSTRACT

The invention is directed to a method for treating ulcers, including ulcers of the lower leg, by topical plus oral administration of proanthocyanidins to a patient.

### FIELD OF THE INVENTION

The invention is directed to novel methods for the treatment of ulcers by a combination of oral and topical administration of proanthocyanidins.

### BACKGROUND

The ulcus of the lower legs, ulcus cruris, represents a serious challenge for medicine, especially in case of diabetic patients. Ulcers of the lower legs are formed mainly as a consequence of chronic venous insufficiency or as a complication of diabetic macroangiopathy. Healing of ulcers is difficult because insufficient or absent circulation blocks transport of oxygen and nutrients to the cells. As a result undernourished cells die and necrosis of tissue develops. The lack of circulation also blocks the removal of cell debris and further impeding normal healing processes difficult. Without the skin barrier, the surface of the ulcus is open for infections, which add to the treatment problems.

Ulcers are different from wounds because whereas normal wounds heal spontaneously over a certain period of time, ulcus cruris, once started, tends to increase in size and wound depth instead of healing. The defective circulation associated with ulcers causes malnutrition and finally necrosis of the tissue. This in turn, causes a progression of the ulceration which cannot be compensated by the normal processes of skin repair.

Current treatment of ulcus of the lower legs involve efforts to promote the normal healing process and to prevent or stop an infection of the open wound. In addition, special exercises are often recommended to stimulate circulation of blood in the lower legs. A vast selection of topical formulations is directed to treatment of ulcus cruris. The products are in most cases combinations of bacteriostatic or bactericidal drugs, vitamins, herbal constituents, absorbing powders, proteolytic enzymes and others.

As a first step to slow down the process of ulceration one can try to treat the underlying diseases—chronic venous insufficiency or diabetes—to prevent a further deterioration of circulation inside the lower legs. Even when a progression of damage of vascular vessels could be stopped by the treatment, the existing pathological changes in blood vessels are irreversible, so that an improvement in healing process is not expected.

Proanthocyanidins are biopolymers, occurring in a wide variety of plant materials. A subgroup of proanthocyanidins, the procyanidins, gained considerable interest because of their biological effects. The procyanidins consist of catechin and epicatechin subunits and form chains of various lengths and binding characteristics. Procyanidins are common constituents of human nutrition, they are found for example in apples, grapes, cola nuts, sorghum, berries, cacao, and many other vegetable extracts.

Proanthocyanidins, especially proanthocyanidins in the form of Pycnogenol®, have been described in numerous references including, for example, Passwater, R. A. The New Superantioxidant Plus, Keats Publishing Inc., New Canaan, Conn. USA, 1992, Passwater, R. A., All About Pycnogenol, Avery Publishing Group, Garden City Park, N.Y., 1998, Passwater, R. A. and Kandaswami, C., Pycnogenol The Super Protector Nutrient, Keats Publishing Inc., New Canaan, Conn. USA, 1994, Passwater, R. A. Pycnogenol for Superior Health, McCleery and Sons Publishing, Fargo, N.D. USA, 2001, and Passwater, R. A. Pycnogenol for Superior Health, Editions Stylum, Switzerland, 2001.

Topical application of procyanidin-containing combinations to treat skin disorders are described in various U.S. patents.

U.S. Pat. No. 5,470,874 describes the use of topically applied mixtures of vitamin C and proanthocyanidins for human skin care. According to the inventor the combination of vitamin C and proanthocyanidins achieves synergy and exponential effectiveness of the free radical scavenging effects of both substances (1, 13-16). Reference is made to the compositions having collagen preservation and collagen repair properties (2). However, no experimental evidence or discussion was presented to show that proanthocyanidins alone is effective in repairing collagen tissue. No ulcer healing is mentioned in U.S. Pat. No. 5,470,874.

U.S. Pat. No. 5,666,365 describes the use of formulations containing coumarins and, optionally, proanthocyanidins, to increase the density of skin capillaries. The wound healing effect of the combination is interpreted in a way that coumarins increase the proanthocyanidin absorption at the topical level. The patent does not teach or suggest the use of proanthocyanidins alone for wound or ulcer healing.

U.S. Pat. No. 5,972,999 protects the use of a complex combination consisting of (1) a sugar compound, (2) an antioxidant, (3) an amino acid and (4) a transition metal to repair skin for the prevention and treatment of wrinkles and other skin disorders. The patent does not teach or suggest the use of proanthocyanidins alone for wound healing or ulcer healing.

Blaszo et al., disclosed that burn wounds healed in a shorter time compared to controls when treated with a gel containing 1 to 5% of a proanthocyanidin-rich extract (Pycnogenol®). However, the authors did not investigate and did not report the effects of a combination of oral and topical treatment and did not discuss the effects of proanthocyanidins on ulcers.

WO 01/05397A1 describes the inhibition of matrix-metallo-proteases by procyanidin-oligomers isolated from genus ulmus. No in-vivo tests were reported, nevertheless, the authors alleged that the procyanidin oligomers isolated from Ulmus genus provide a method to promote wound and burn healing.

None of the references disclose or suggest the use of a combination of oral and topical proanthocyanidins for ulcer healing. Thus, there is an unmet need for a method for the treatment of ulcers such as the ulcers of the lower legs—ulcus cruris.

### BRIEF DESCRIPTION OF THE INVENTION

One embodiment of the invention is directed to a method for preventing or treating ulcers of the lower leg in a patient. In the method, a composition comprising proanthocyanidins is administered both topically and orally to a patient in need of treatment. The composition for oral administration may be the same or different from the composition for topical administration. The method when applied to a patient with leg ulcers can reduce the size or number of ulcers.

The topical composition may comprise proanthocyanidins in a concentration between 0.1 to 100%. The topical composition may be a powder, a gel, an ointment, a lotion, a cream, an oily solution, a suspension, or a semi-solid. The oral composition may be in the form of pills, drinks, powders, food additives, powders, capsules, time-release-capsules, slow-release formulations and other formulations for oral intake.

The proanthocyanidins may be from a proanthocyanidins-rich plant extract comprising 10% to 100% proanthocyanidins. In addition, the proanthocyanidins compositions may further comprise an antiseptic agent, an anti-inflammatory agent, an analgesic agent, a wound healing agent or a combination thereof.

The preferred dosage for the oral or topical administration is between 20 mg to about 10 grams per patient per day, such as between 20 mg to 2 grams per patient per day. This dosage may be administered once a day or multiple times a day. The number of administrations per day may be, for example, 2, 3, 4, 5, 6 or more. That is, the administration is applied on a periodic basis each day over the course of a treatment period.

The treatment period may be at least 30, 42, 60, 90, or 120 days.

Proanthocyanidins may be derived from synthesis or from plant materials. Nonlimiting examples of such materials include grape seeds, grape skin, pine barks, ginkgo leaves, peanuts, and cocoa beans, tamarind, tomato, peanut, almond, apple, cranberry, blueberry or tea leaves. In a preferred embodiment, the proanthocyanidins are from pine bark, such as for example, from Pinus pinaster bark.

### BRIEF DESCRIPTION OF THE DRAWING

FIG. 1 depicts variation of size of venous ulceration with proanthocyanidins (Pycnogenol®) treatments.

### DETAILED DESCRIPTION OF THE INVENTION

In experiments to determine a course of treatment for ulcers, we have found, to our surprise, that proanthocyanidins alone are able to accelerate ulcer healing without the use of additional active ingredients. These additional ingredients, which are now found to be unnecessary, include vitamin C and coumarins.

The term “treating” in its various grammatical forms in relation to the present invention refers to preventing, curing, reversing, attenuating, alleviating, ameliorating minimizing, suppressing, or halting the deleterious effects and symptoms of ulcers such as leg ulcers. Nonlimiting examples of lower leg ulcers symptoms include destruction, deficit of skin caused by necrosis, desquamation and melting of skin. Moreover, skin ulcers can be caused or exacerbated by wounds, decubitus, scalding, frostbite, or operation wounds, skin infections (e.g. skin mycosis, psoriasis, varicella, tinea pedis, tinea corporis, pimple etc.) and underlying ailments such as diabetes.

Proanthocyanidins designate a group of flavanoids that includes the subgroups procyanidins, prodelphinidins and propelargonidins. Proanthocyanidins are homogeneous or heterogeneous polymers consisting of the monomer units catechin or epicatechin, which are connected either by 4-8 or 4-6 linkages, to the effect that a great number of isomer proanthocyanidins exist. Typically, the proanthocyanidins oligomers have a chain length of 2-12 monomer units. Proanthocyanidins may be synthesized or extracted from a plant material. Nonlimiting examples of plant material sources of proanthocyanidins include grape seeds, grape skin, pine barks, ginkgo leaves, peanuts, and cocoa beans, tamarind, tomato, peanut, almond, apple, cranberry, blueberry, tea leaves.

Every plant species has its own unique proanthocyanidins mix. In a preferred embodiment, the proanthocyanidins are derived from pine bark. A well-known product containing proanthocyanidins, which is available in trade as a preparation of a food supplement under the name Pycnogenol®, is a registered trademark belonging to Horphag Research, Ltd. The Pycnogenol® food supplement contains approximately 70-80% of proanthocyanidins and is a complex mixture of phenolic substances. Besides proanthocyanidins and its monomeric unit catechin, Pycnogenol® contains taxifolin and a wide range of phenolic acids, e.g. free acids like p-hydroxybenzoic acid, protocatechic acid, vanillic acid, caffeic acid and ferulic acid as well as its glucosides and glucose esters (21). Most of the positive effects of Pycnogenol® are attributed to its antioxidant qualities.

While the proanthocyanidins of this disclosure are not limited to those derived from any source, one preferred source of proanthocyanidins is French maritime pine bark extract (Pycnogenol®), obtained from Pinus pinaster, syn. Pinus maritima. It is understood that Pycnogenol® may be used wherever this disclosure refers to proanthocyanidins.

We have found, unexpectedly, the oral administration of proanthocyanidins (French maritime pine bark extract, Pycnogenol®) was highly effective in reducing ulcer size compared to controls in patients with chronic venous insufficiency. We further report that a combination of oral and topical application of proanthocyanidins provides additional synergistic benefits not found by oral or topical administration alone. When patients were treated orally with a proanthocyanidin-rich extract and wounds were covered with bandages containing the same proanthocyanidin-rich extract as powder on the surface, ulcer size decreases faster compared to oral treatment. After 6 weeks a total healing of ulcus cruris could be obtained, ulcer size shrank to zero.

Furthermore, we found that the methods of the invention is effective for treatment of diabetic ulcers—such as diabetic ulcers of the lower leg. Diabetes influences not only the metabolism but also the healing process of wounds and the circulation. Patients with diabetes have to take special care of ulcers because healing is very difficult to obtain. Unexpectedly, the intake of proanthocyanidins accelerated healing of ulcus cruris also in diabetic patients compared to controls in a significant manner. Even more effective was the combined oral and topical treatment with the proanthocyanidin-rich extract. After 6 weeks of combined treatment, ulcer size shrank to 2 mm from a starting ulcer area of 52 mm.

The examples of this disclosure demonstrate that ulcus cruris can be cured by a method combining the topical and oral application of proanthocyanidin rich extracts. The method of the invention is also effective for treatment of ulcers in diabetic patients. An explanation for these pronounced effects of the proanthocyanidins could be the combined effect of an improved microcirculation together with the diverse anti-inflammatory actions.

Selection of Patients and Experimental Methods

### EXAMPLE 1

Patients with severe CVI (chronic venous insufficiency) causing ulcerations were included in this study. The definition of CVI and its assessment both with ultrasound and ambulatory venous pressures (AVP) measurements have been discussed before.

All subjects included into the study had an AVP>55 mmHg with a refilling time shorter than 8 seconds. The incompetence (also shown by duplex evaluation) was mainly deep with very limited superficial incompetence. Duplex also indicated that there was no obstruction of recent thrombosis. All subjects hade previous history of thrombosis.

The ulceration was present in their history for the first time and the lesion had been present for at least 2 months.

Exclusion criteria were any clinical disease requiring treatment, severe bone/joint problems or limited mobility, uncontrolled diabetes severe hypertension obesity recent thrombosis (less than 6 months), the presence of thrombi.

A thrombosis was defined when a non-compressible vein clot was observed by ultrasound (both in the deep system and in the superficial veins). The presence of inflammation of a superficial vein, without thrombosis, was defined phlebitis.

An exercise plan was presented to all included subjects in an educational video explaining venous thrombosis and its prevention. It consisted of mild exercise (standing and moving legs for 5-10 minutes every hour) and avoiding long standing and sitting periods. Compression stockings (25-30 mmHg at the ankle) were used during the study.

Pycnogenol Administration

Oral application: Subjects received 1 capsule of 50 mg Pycnogenol®, three times daily. Local application: After careful washing of the ulcer area and cleaning with diluted mild local disinfectant (Citrosil) the ulcerated area was dried with paper tissue and the powder from capsules (2 for each ulcers, 100 mg Pycnogenol®) was applied in a fine layer (dispersing it on the ulcer surface).

Paper-non allergic dressing was placed over the ulcer and a layer of Tensoplast (elastic-adhesive bandage) was applied to control edema. Compression stockings were finally applied (20-25 mmHg of pressure at the ankle). The local treatment was replaced every 2 days. The treatment and follow up period was 6 weeks.

A microcirculatory evaluation was also performed (at inclusion and at 6 weeks) measuring the transculaneous PO2 and PCO2 with a combined measurement (skin at 44°) after stabilization and capillarisation of the area.

The measurements were made at least at 1 cm from the ulcer edge, in non-inflamed or infected area where the skin was intact.

In venous ulcers the increase in skin PCO2 is a negative aspects indicating slower and altered perfusion. This is usually associated with a decrease on skin PO2 indicating a decreased level of oxygenation and slower, less efficient perfusion.

The target, considering the microcirculation is to improve (increasing it) PO2 also decreasing PCO2.

As discussed above, proanthocyanidins may be in the form of Pycnogenol® which is an acqueous, dry extract from the bark of the French maritime pine. It is a natural blend of constant proportions of bioflavonoids including catechin, epicatechin, taxifolin, oligomeric proanthocyanidnins and phenolic acids as ferulic acid and caffeic acid. Clinical studies in thousand of patients have shown, very rarely, only very mild, temporary side effects (20-23).

In this study tolerability and compliance were evaluated by direct questioning particularly considering gastrointestinal problems, systemic and local skin alterations, signs of allergic reaction and any other manifestation.

A variation in symptomatic score (0 no symptoms, 10 very severe signs/symptoms) was also recorded at the beginning and at the end of the study.

Results

We included 18 subjects; 16 completed the study; 2 subjects were lost to follow-up for logistical problems (Table 1).

The age and male to female distributions were comparable among the three groups.

The reduction in ulcerated area (Table 2) was more relevant in patients using both systemic and local treatment.

No side effects were observed.

The more important decrease in the ulcerated are was also associated to marked improvement in signs and symptoms as shown by the symptomatic score variations in those using both local and systemic treatment.

Table 3 shows the variations in microcirculatory parameters (transcutaneous PO2 and PCO2 in mmHg; mean and SD).

A significant improvement—an increase in skin PO2 and a decrease in skin PCO2—is associated to the faster healing observed in the oral+local treatment group. The improvements in the placebo group are due only to compression which is usually quite effective in these patients.

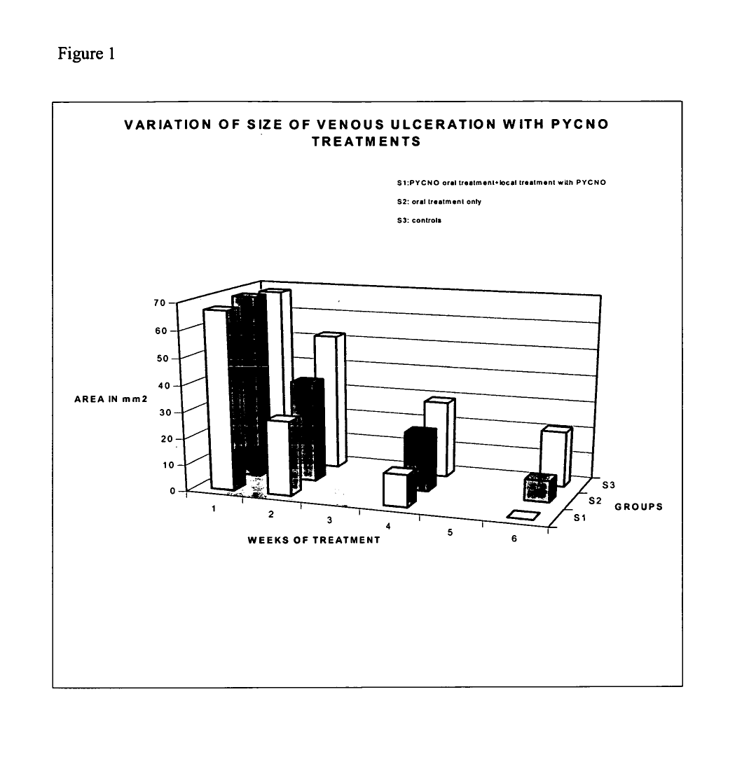

The results of these experiments are summarized in FIG. 1. In FIG. 1, the area of the ulcer (in mm²) is plotted against time. Oral treatment of proanthocyanidins (in the form of Pycnogenol) has an effect on ulcer reduction (See S2). However, a combination of oral and topical treatment of proanthocyanidins leads to the virtual elimination of ulcer after 6 weeks (See, S1) relative to the control (i.e., no treatment. See, S3).

Discussion: Treatment of CVI and Venous Ulcerations

As skin flux is increased PO2 decreased and PCO2 increased (the result of chronic increase in venous pressure, transmitted at the venous end of the microcirculatory levels) the aim of treatment of CVI and venous microangiopathy is to reverse these changes.

Surgical treatment or occlusion/obliteration of incompetent veins with compression sclerotherapy reverse in time these chronic microcirculation alterations and the increased ulceration risk.

Compression therapy in patients with CVI temporarily reduces hyperperfusion and edema and elastic compression is widely and effectively used as a conservative measure.

Elastic compression does not treat venous hypertension but is very effective in controlling it during the daily hours of working and standing. Elastic stockings in clinical practice actually have a double important effect: (a): they decrease venous pooling and micropooling preventing edema formation and eventually improving skin perfusion; (b): they protect skin with venous microaniopathy from micro-trauma which are usually the starting event of an ulceration.

In areas of VHM (venous hypertensive microangiopathy) the unbalanced nutritional status makes skin very delicate and unable to cope with microtrauma which usually occur in normal daily activity. However elastic compression is not always bearable (i.e. in higher temperature or warm climates) and older subjects may find difficult to put on stronger stockings.

The best combination of conservative treatment (i.e. elastic stockings, exercise, controlling the role of risk factors) and more invasive therapy (surgery+sclerotherapy) should be found according to patients' needs and clinical picture as the same, standardised treatment is not possible for all patients. Surgery, when possible, is more effective (but more complex and expensive) than sclerotherapy. Even compression and the injection of a few veins can decrease venous hypertension and produce venous ulcers healing.

However some ulcerations are difficult to heal due to a combination of venous hypertension, infection, edema and stasis.

All methods and drugs controlling edema may have a important effect in treating VHM and its consequences. Real venous ulcers are caused by increased venous pressure while most ulcers seen in ‘venous ulcers’ clinics are mainly due to social problems, lack of exercise, neglect, obesity, stasis and constitute a social problem more than a medical one. Ulcers are therefore difficult to heal and tend to recur in several patients and conditions.

In this study the application of Pycnogenol on the venous ulcer area contributed to faster healing possibly by acting on local edema and also on bacterial proliferation in the ulcerated area. There is a possibility that Pycnogenol may also act as a collagen modulator promoting growth factors in the area. The local action, combined with the systemic action of Pycnogenol may offer an important new solution to the difficult treatment of venous ulcerations.

All patents, patent applications, and references cited in this disclosure are incorporated by reference in their entirety.

TABLE 1
DETAILS OF SUBJECTS. Three groups were included. One group was
treated with Pycnogenol (combined oral and topic administration), one
with oral Pycnogenol only and one with placebo. After 6 weeks of
the 18 subjects were included and 2 were lost (one in the placebo and
one in the oral Pycnogenol group).

| | A | | | | | |
|---|---|---|---|---|---|---|
| | PYCNO | B | | | | |
| | OR + | PYCNO | C | | | |
| WEEK | LOCAL | ORAL | PLACEBO | TOTAL | AGE(SD) | M:F |
| 1 | 6 | 6 | 6 | 18 | 56.6 | 10:8 |
| 2 | 6 | 5 | 6 | 17 | | 9:8 |
| 3 | — | — | — | | | |
| 4 | 6 | 5 | 6 | 17 | | |
| 5 | — | — | — | | | |
| 6 | 6 | 5 | 5 | 16 | | 8:8 |

TABLE 2
variations in the area of ulceration (mm 2).
Variation in the symptomatic score (0 no symptoms,
10 very severe signs/symptoms).

| | A | B | | |
|---|---|---|---|---|
| | PYCNO | PYCNO | C | |
| WEEK | OR + LOCAL | ORAL | PLACEBO | P value |
| 1 | 67 | 69 | 68 | n.s. |
| | 8 | 9 | 8 | n.s. |
| 2 | 28** | 38* | 52 | <0.05 |
| 3 | — | — | — | |
| 4 | 12** | 22** | 29* | <0.025 |
| | 3 | 6 | 7 | <0.05 |
| 5 | — | — | — | |
| 6 | 0*** | 8** | 21* | <0.025 |
| | 0 | 4 | 6 | <0.05 |

TABLE 3
variations in microcirculatory parameters (transcutaneous PO2 and
PCO2 in mmHg; mean and SD). An improvement is indicated by an
increase in skin PO2 and by a decrease in skin PCO2. Measuremente
were made on intact skin, at least 1 cm from the edge of the ulceration
(Kontron, Combisensor).

| | | A | B | | |
|---|---|---|---|---|---|
| | | PYCNO | PYCNO | C | |
| WEEK | | OR + LOCAL | ORAL | PLACEBO | P value |
| 1 | PO2 | 47; SD 4 | 48.1; 5 | 48.2; 4 | n.s. |
| | CO2 | 33; 2 | 32; 3 | 31; 2 | n.s. |
| 6 | PO2 | 58.2; 3 | 55; 4 | 48.1; 3 | <0.05 |
| | PCO2 | 27; 3 | 29; 2 | 29; 3 | <0.05 |

### REFERENCES

1. Belcaro G, Bollinger A, Hoffman U, Nicolaides An. Laser Doppler, Med-Orion, London 1996.
2. Belcaro G, Nicolaides An, Stansby G. The Venous Clinic. Imperial College Press, London 1999
3. Belcaro G, Nicolaides An. Noninvasive Investigations In Vascular Disease. Imperial College Press, London 2000
4. Belcaro G, Nicolaides An, Veller M. Venous Disorders. A Manual Of Diagnosis And Treatment. Saunders, Cambridge, 1995
5. Belcaro G. Laser Doppler in venous hypertension. PhD. Thesis University of London. UCL
6. Bollinger A, Leu A J, Hoffman U, Franzeck U K. Microvascular changes in venous disease: an update. Angiology 1997; 48:27-32.
7. Nicolaides A N, Abenhaim L, Akeson H, Agus G, Allegra G, Andreoni G M, Balas P, Becker F, Belcaro G, Belz G G, Bergan J, Bergqvist D et al. Investigation of chronic venous insufficiency. A consensus statement. Circulation 2000;102: e126-63.
8. Rohdewald P. A review of the French maritime pine bark extract (Pycnogenol®), a herbal medication with a diverse clinical pharmacology. Int J Clin Pharmacol & Ther 2002,40,4: 158-68.
9. Petrassi, C., Mastromarino, A. and Spartera, C. Pycnogenol® in chronic venous insufficiency. Phytomedicine, 7(5): 383-88, 2000.
10. Putter M, Grotemeyer K H M, Würthwein G, Araghi-Niknam R R, Watson R R, Hosseini S, Rohdewald P. Inhibition of smoking-induced platelet aggregation by aspirin and Pycnogenol. Thrombosis Research 95:155-61, 1999.
11. Schmidtke. I. and Schoop, W. Le pycnogenol : Therapeutique medicamenteuse de I'oedeme statique. Journal Suisse de medecine globale. 3/95: 114-115, 1995.
12. Belcaro G, Nicolaides A N, Veller M. Venous Disorders: a manual of diagnosis and treatment. Saunders, Cambridge, 1996.
13. Belcaro G, Nicolaides A N. Noninvasive investigations in vascular disease. Imperial College Press, London, 2001.
14. Belcaro G. Nicolaides A N, Stansby G. The Venous Clinic. Imperial College Press, London, 1999.
15. Thulesius O. Chronic venous insufficiency and varicose veins physiopathological sight points. Lakartidningen. 80 (17):1791-801, 1983.
16. Cesarone M R, Belcaro G, Nicolaides A N et al. The edema tester in the evaluation of swollen limbs in venous and lymphatic disease. Panmin Med 1999;41:10-14.
17. Belcaro G, Bollinger A, Hoffinan U, Nicolaides A N (eds). Laser-Doppler. Med-Orion, London 1995.
18. Nicolaides A N. Investigation of chronic venous insufficiency: a consensus statement. Circulation 102: 126-63,2000.
19. Dalen J E, Hirsh J, Guyatt G H. Sixth ACCP Consensus Conference on Antithrombotic Therapy. Chest 119;1 (Suppl), 2001.
20. Prevention Of Venous Thromboembolism. International Consensus Statement. Edition 2002; Med-Orion, London, 2002.
21. Rohdewald P. 2002. A review of the French maritime pine bark extract (Pycnogenol®), a herbal medication with a diverse clinical phamacology. Int J Clin Pharmacol Therap, 40 (4):158-168.21.

We claim:

1. A method of preventing or treating ulcer of the lower leg in a patient comprising the following steps in any order:
administering topically to said patient an effective amount of a first composition comprising proanthocyanidins; and
administering orally to said patient an effective amount of a second composition comprising proanthocyanidins,
wherein said ulcer of the lower leg is prevented or treated.

2. The method of claim 1 wherein the first composition is in a topical form selected from the group consisting of powders, gels, creams, lotions, aqueous alcoholic solutions, a suspension, a semi-solid.

3. The method of claim 1 wherein the second composition is in an oral form selected from the group consisting of pills, drinks, powders, food additives, powders, capsules, time-release-capsules.

4. The method of claim 1 wherein said proanthocyanidins are a proanthocyanidins-rich plant extract comprising 10% to 100% proanthocyanidins.

5. The method of claim 1 wherein said topical composition further comprises an antiseptic agent, an anti-inflammatory agent, an analgesic agent, a wound healing agent or a combination thereof.

6. The method of claim 1 wherein about 20 mg to about 10 grams per patient per day of proanthocyanidins are administered topically.

7. The method of claim 1 wherein about 20 mg to about 10 grams per patient per day of proanthocyanidins are administered orally.

8. The method according to claim 1 wherein said administration is applied on a periodic basis each day over the course of a treatment period.

9. The method of claim 1 wherein said administrating comprises administering said proanthocyanidins daily for a period of at least 30 days, at least 42 day, at least 60 days, at least 90 days or at least 120 days.

10. The method of claim 1 wherein the proanthocyanidins are provided as an extract from a plant material.

11. The method of claim 10 wherein the proanthocyanidins are extracted from pine bark.

12. The method of claim 11 wherein said pine bark is from Pinus pinaster.
